(19) United States
(12) Patent Application Publication
(10) Pub. No.: US 2007/0104747 A1
(43) Pub. Date: May 10, 2007

(54) Cosmetic composition comprising at least one anti-dandruff agent and also oxyethylenated sorbitan monolaurate, and cosmetic treatment process using said composition

(76) Inventors: Virginie Masse, Clichy (FR); Sandrine Decoster, Saint-Gratien (FR); Beatrice Thomas, Issy les Moulineaux (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/589,069

(22) Filed: Oct. 30, 2006

Related U.S. Application Data
(60) Provisional application No. 60/736,297, filed on Nov. 15, 2005.

(30) Foreign Application Priority Data
Oct. 28, 2005 (FR) 05 11095

(51) Int. Cl. A61K 31/7048; A61K 31/7034; A61K 33/24; A61K 33/04; A61K 31/555; A61K 31/496

(52) U.S. Cl. 424/401; 424/702; 424/725.1; 424/617; 514/28; 514/254.07; 514/184; 514/35

(57) ABSTRACT

Disclosed herein is a cosmetic composition comprising, in an aqueous medium, at least one anti-dandruff agent other than 2-pyridone derivatives, and at least one oxyethylenated sorbitan monolaurate comprising from 2 to 25 oxyethylene units. The anti-dandruff agent may be chosen, for example, from zinc pyrithione, salicylic acid, selenium disulphide, and mixtures thereof. Also disclosed herein is a cosmetic treatment process comprising applying the composition to the hair.

This application claims benefit of U.S. Provisional Application No. 60/736,297 filed Nov. 15, 2005, the contents of which are incorporated herein by reference. This application also claims benefit of priority under 35 U.S.C. § 119 to French Patent Application No. FR 05 11095, filed Oct. 28, 2005, the contents of which are also incorporated herein by reference.

Disclosed herein is a cosmetic composition comprising at least one anti-dandruff agent other than 2-pyridone derivatives, and at least one oxyethylenated sorbitan monolaurate comprising from 2 to 25 oxyethylene units. Also disclosed herein is a process. for the cosmetic treatment of the hair and/or of the scalp using the cosmetic composition.

Numerous anti-dandruff compositions have been proposed in the prior art with a view to combating the formation of dandruff.

For instance, International Patent Application Publication No. WO 2004/089318 describes shampoo compositions having an improved detergent and anti-dandruff action. These compositions contain:
(a) from 5 to 50% by weight of a detergent surfactant,
(b) from 0.1 to 4% by weight of an anti-dandruff agent,
(c) from 0.1 to 50% by weight of a polyoxyethylenated derivative of an ester of a fatty acid and of sorbitan, and
(d) at least 20% by weight of water.

The compound (c) is derived from an ester of sorbitan and of a C₁₄ to C₁₈ fatty acid such as palmitic acid, stearic acid, and oleic acid. It corresponds, for example, to products such as those sold under the names TWEEN 40, TWEEN 60, TWEEN 61, and TWEEN 85 by the company Uniqema.

European Patent Application EP 0 117 135 describes detergent compositions having improved antimicrobial and anti-dandruff properties. These compositions comprise at least one anionic or amphoteric surfactant, at least one nitrogenous polymer, and at least one water-soluble non-particulate substance, such as anti-dandruff agents, sun protection agents, and insecticidal agents.

The examples present anti-dandruff shampoo compositions containing, as anti-dandruff agent, the products called OCTOPIROX and OMADINE MDS. Some of these products also contain oxyethylene sorbitan monolaurate comprising 40 OE, or oxyethylenated sorbitan monolaurate comprising 80 OE.

Finally, International Patent Application Publication No. WO 03/096998 describes shampoo compositions which prevent the formation of dandruff. These compositions contain, as active ingredient, extracts of Camelia sinensis, or the active ingredients thereof of the polyphenol type (for example, catechins, flavonoids). These compositions can also comprise additional anti-dandruff agents, and anionic, nonionic, amphoteric, zwitterionic, and/or cationic surfactants. Among nonionic surfactants, mention may be made of esters of fatty acids and of sorbitan, and polyoxyethylenated derivatives thereof comprising from 1 to 30, for instance, from 5 to 10 oxyethylene units.

The compositions of the prior art such as those described above all have the objective of improving the anti-dandruff effect and, optionally, improving detergence. Moreover, they do not always exhibit satisfactory properties for use, for example, in terms of viscosity and of foam quality.

Individuals who use anti-dandruff shampoos generally have sensitive scalps. In addition, the frequent use of shampoos of this type has a tendency to accentuate discomforting reactions such as redness, itching, and stinging, etc.

Thus, it is desirable to provide compositions which exhibit better skin tolerance while at the same time conserving good anti-dandruff effectiveness and good properties for use.

The present inventors have now discovered that, by combining at least one surfactant with at least one anti-dandruff agent other than 2-pyridone derivatives, it is possible to formulate a cosmetic composition which exhibits an excellent anti-dandruff activity while at the same time making it possible to decrease phenomena of discomfort on the scalp and having improved properties for use, for example, in terms of viscosity.

The compositions according to the present disclosure, which comprise at least one anti-dandruff agent other than 2-pyridone derivatives, combined with at least one oxyethylenated sorbitan monolaurate comprising from 2 to 25 oxyethylene units, may exhibit excellent skin tolerance.

Moreover, in addition to their improved anti-dandruff effectiveness, the compositions according to the present disclosure may exhibit both improved cleansing and cosmetic properties.

Disclosed herein, therefore, is a cosmetic composition comprising, in an aqueous medium, at least one anti-dandruff agent other than 2-pyridone derivatives, and at least one oxyethylenated sorbitan monolaurate comprising from 2 to 25 oxyethylene units.

Also disclosed herein is a cosmetic treatment process comprising applying said composition to keratin fibers.

Other subjects and characteristics, aspects, and advantages of the present disclosure will emerge even more clearly upon reading the description and the examples which follow.

According to the present disclosure, the cosmetic composition comprises, in an aqueous medium, at least one anti-dandruff agent other than 2-pyridone derivatives, and at least one oxyethylenated sorbitan monolaurate comprising from 2 to 25 oxyethylene units.

As used herein, and as understood in the prior art, the expression “oxyethylenated compound with X OE” denotes an oxyethylenated compound comprising X units of oxyethylene per molecule.

Anti-Dandruff Agents

The anti-dandruff agents which can be used in the composition disclosed herein include compounds known to those skilled in the art, with the exclusion of 2-pyridone derivatives.

The at least one anti-dandruff agent may be any active agent which can be used to prevent the appearance of dandruff, to decrease the amount thereof, and/or to cause it to completely disappear. Thus, the at least one anti-dandruff agent may be chosen, for example, from antifungal and/or antibacterial agents.

For example, the at least one anti-dandruff agent may be chosen from:

1) pyridinethione salts, for instance, the calcium, magnesium, barium, strontium, zinc, cadmium, tin, and zirconium salts. In at least one embodiment, the anti-dandruff agent may be the zinc salt of pyridinethione, such as the product sold under the name ZINC OMADINE by the company Olin;

2) trihalocarbamides of formula: 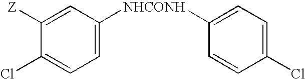
wherein Z is chosen from halogen atoms such as chlorine and C₁-C₄ trihaloalkyl groups such as CF₃;

3) triclosan, represented by the formula:

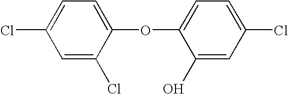

4) azole compounds such as climbazole, ketoconazole, clotrinazole, econazole, isoconazole, and miconazole;

5) antifungal polymers such as amphotericin B and nystatin;

6) selenium sulphides, for example, those of formula SₓSe₈₋ₓ, wherein x is a number ranging from 1 to 7;

7) other anti-dandruff agents, which comprise sulphur in its various forms, cadium sulphide, allantoin; coal tar, wood tar, and derivatives thereof, for instance, oil of cade; salicylic acid; undecylenic acid; fumaric acid; and allylamines such as terbinafine.

According to at least one embodiment, the at least one anti-dandruff agent may be chosen from zinc pyrithione, salicylic acid, selenium disulphide, and mixtures thereof.

The at least one anti-dandruff may be present in the composition disclosed herein in an amount ranging from 0.001 to 10% by weight, for example, from 0.1 to 5% by weight, or from 0.2 to 2% by weight, relative to the total weight of the composition.

Surfactants

The composition of the present disclosure comprises at least one surfactant chosen from oxyethylenated sorbitan monolaurates comprising from 2 to 25 oxyethylene units.

In at least one embodiment, the composition according to the present disclosure comprises at least one oxyethylenated sorbitan monolaurate comprising from 2 to 10 oxyethylene units.

According to another embodiment, the composition according to the present disclosure comprises oxyethylene sorbitan monolaurate with 4 OE. This compound is also known under the name polysorbate 21 and is sold, inter alia, under the name TWEEN 21 by the company UNIQEMA.

This compound may be used alone, or as a mixture with other oxyethylenated derivatives, for instance, those of sorbitan monolaurate. For example, in at least one embodiment, it is possible to use oxyethylene sorbitan monolaurate comprising 4 OE, as a mixture with oxyethylene sorbitan monolaurate comprising 20 OE.

The at least one oxyethylenated sorbitan monolaurate comprising from 2 to 25 oxyethylene units may be present in the composition in an amount of at least 0.5% by weight relative to the total weight of the composition, for example, from 0.5 to 10% by weight, from 2 to 9% by weight, or from 3 to 8% by weight, relative to the total weight of the composition.

Optional Ingredients

The composition according to the present disclosure may also comprise at least one fatty alcohol comprising at least 12 carbon atoms, for example, from 12 to 30 carbon atoms, or, in a still further embodiment, from 16 to 24 carbon atoms.

Non-limiting examples of such fatty alcohols include cetyl alcohol, stearyl alcohol, arachidyl alcohol, behenyl alcohol, lignoceryl alcohol, ceryl alcohol, montanyl alcohol, and mixtures thereof, and in at least one embodiment, behenyl alcohol.

The at least one fatty alcohol may be, in at least one embodiment, in the composition in an amount greater than or equal to 0.5% by weight, for instance, ranging from 0.5% to 10% by weight, or, in a still further embodiment, from 1 to 5% by weight, relative to the total weight of the composition.

The compositions according to the present disclosure may be in a form chosen from shampoos and compositions to be applied before or after a shampoo, the latter being in a form chosen from more or less thickened lotions, gels, and emulsions.

The compositions of the present disclosure may also comprise at least one surfactant chosen from anionic, cationic, amphoteric, and nonionic surfactants different from the oxyethylenated sorbitan monolaurate comprising from 2 to 25 oxyethylene units.

Examples of suitable anionic surfactants include salts, for instance, alkali metal salts such as sodium salts, ammonium salts, amine salts, and aminoalcohol salts, and alkaline earth metal salts, for example, magnesium salts of alkyl sulphates, alkyl ether sulphates, alkylamido ether sulphates, alkylaryl polyether sulphates, monoglyceride sulphates, alkylsulphonates, alkylamidesulphonates, alkylarylsulphonates, α-olefin sulphonates, paraffin sulphonates, alkylsulphosuccinates, alkyl ether sulphosuccinates, alkylamidesulphosuccinates, alkylsulphoacetates, acylsarcosinates, and acylglutamates, wherein the alkyl and acyl-groups of these compounds comprise from 6 to 24 carbon atoms and the aryl group is chosen from phenyl and benzyl groups.

Other examples include, but are not limited to, C₆₋₂₄ alkyl monoesters of polyglycoside dicarboxylic acids, such as alkyl glucoside citrates, alkyl polyglycoside tartrates, and alkyl polyglycoside sulphosuccinates, alkylsulphosuccinamates, acylisethionates, and N-acyltaurates, wherein the alkyl or acyl group of these compounds comprises from 12 to 20 carbon atoms.

Further non-limiting examples of anionic surfactants which can be used in the compositions of the present disclosure include acyl lactylates, the acyl group of which comprises from 8 to 20 carbon atoms.

Alkyl-D-galactosideuronic acids and salts thereof, and polyoxyalkylenated (C₆₋₂₄ alkyl)ether carboxylic acids, polyoxyalkylenated (C₆₋₂₄ alkyl)(C₆₋₂₄ aryl) ether carboxylic acids, polyoxyalkylenated (C₆₋₂₄ alkyl)amido ether carboxylic acids, and salts thereof, for instance, those comprising from 2 to 50 ethylene oxide units, and mixtures thereof may also be used.

In at least one embodiment, the at least one anionic surfactant may be chosen from alkyl sulphates, alkyl ether sulphates, and mixtures thereof, for example in the form of alkali metal and alkaline earth metal salts, ammonium salts, amine salts, and aminoalcohol salts.

The at least one anionic surfactant may be present in the composition in an amount ranging from 0.1 to 50% by weight, for example, from 4 to 20% by weight relative to the total weight of the composition.

Examples of additional nonionic surfactants are described, for example, in “Handbook of Surfactants” by M. R. PORTER, publisher Blackie & Son (Glasgow and London), 1991, pp 116-178. The at least one non-ionic surfactant may be chosen from alcohols, alpha-diols, (C₁₋₂₀)alkylphenols, and polyethoxylated, polypropoxylated, and polyglycerylated fatty acids comprising a fatty chain comprising, for example, from 8 to 18 carbon atoms, it being possible for the number of ethylene oxide or propylene oxide groups to range from 2 to 50 and it being possible for the number of glycerol groups to range from 2to 30.

The at least one additional non-ionic surfactant may also be chosen from condensates of ethylene oxide and of propylene oxide with fatty alcohols; polyethoxylated fatty amides comprising, for example, from 2 to 30 ethylene oxide units, polyglycerylated fatty amides comprising, on average, from 1 to 5 glycerol groups, for instance, from 1.5 to 4, ethoxylated fatty acid esters of sorbitan comprising from 2 to 30 ethylene oxide units, fatty acid esters of sucrose, fatty acid esters of polyethylene glycol, (C₆₋₂₄ alkyl)-polyglycosides, N-(C₆₋₂₄ alkyl)glucamine derivatives, amine oxides such as (C₁₀₋₁₄ alkyl)amine oxides, and N-(C₁₀₋₁₄ acyl)aminopropylmorpholine oxides.

The at least one additional nonionic surfactant may be present in the composition in an amount ranging from 0.01 to 20% by weight, for example, from 0.2 to 10% by weight relative to the total weight of the composition.

The amphoteric or zwitterionic surfactants which may be used in the compositions of the present disclosure may include, for example, derivatives of secondary or tertiary aliphatic amines in which the aliphatic group is a linear or branched chain comprising from 8 to 22 carbon atoms and containing at least one anionic group chosen, for example, from carboxylate, sulphonate, sulphate, phosphate, and phosphonate groups. (C₈₋₂₀)alkylbetaines, sulphobetaines, (C₈₋₂₀ alkyl)amido(C₆₋₈ alkyl)betaines, and (C₈₋₂₀ alkyl)-amido (C₆₋₈ alkyl)sulphobetaines may also be used.

Examples of suitable amine derivatives include, but are not limited to, the products sold under the name MIRANOL®, as described in U.S. Pat. Nos. 2,528,378 and 2,781,354 and classified in the CTFA dictionary, 3rd edition, 1982, under the names amphocarboxyglycinate and amphocarboxypropionate, having the respective structures (II) and (III):
Rₐ—CONHCH₂CH₂—N(R_{b})(R_{c})(CH₂COO⁻⁾ (II )

wherein:
Rₐ is chosen from alkyl groups derived from an acid Rₐ-COOH present in hydrolyzed coconut oil, and heptyl, nonyl, and undecyl groups,
R_{b} is a beta-hydroxyethyl group, and
R_{c} is a carboxymethyl group; and
   Rₐ′—CONHCH₂CH₂—N(B)(B′) (III)

wherein:
B is —CH₂CH₂OX′,
B′ is —(CH₂)_{z}—Y′, wherein z=1 or 2,
X′ is chosen from —CH₂CH₂—COOH and hydrogen,
Y′ is chosen from —COOH and —CH₂—CHOH—SO₃H, and
Rₐ′ is chosen from alkyl groups of an acid Rₐ′—COOH present in coconut oil or in hydrolysed linseed oil, alkyl groups, for example, C₁₇ alkyl groups and their iso forms, and unsaturated C₁₇ groups.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

A non-limiting example of such an amine derivative is the cocoamphodiacetate sold by the company RHODIA under the trade name MIRANOL® C2M concentrate.

In at least one embodiment, the at least one amphoteric or zwitterionic surfactant may be chosen from (C₈₋₂₀ alkyl)betaines, (C₈₋₂₀ alkyl)amido(C₆₋₈ alkyl)betaines, and mixtures thereof.

The at least one amphoteric or zwitterionic surfactant may be present in the composition in an amount ranging from 0.01 to 20% by weight, for example, from 0.5 to 10% by weight relative to the total weight of the composition.

In at least one embodiment, the composition according to the present disclosure may comprise at least one anionic surfactant and at least one amphoteric or zwitterionic surfactant.

In another embodiment, the compositions according to the present disclosure may have a total content of surfactants (anionic, nonionic, and/or amphoteric or zwitterionic surfactants) ranging from 4 to 50% by weight, for example, from 4 to 20% by weight, relative to the total weight of the composition.

Examples of suitable cationic surfactants include, but are not limited to, salts of primary, secondary, or tertiary fatty amines, which are optionally polyoxyalkylenated; quaternary ammonium salts, such as tetraalkylammonium chlorides and bromides, alkylamidoalkyltrialkylammonium chlorides and bromides, trialkylbenzylammonium chlorides and bromides, trialkylhydroxyalkylammonium chlorides and bromides, alkylpyridinium chlorides and bromides; imidazoline derivatives; and amine oxides which are cationic in nature.

The at least one cationic surfactant may be present in the composition in an amount ranging from 0.01 to 10% by weight, for instance, from 0.2 to 5% by weight, or from 0.3 to 3% by weight relative to the total weight of the cosmetic composition.

The compositions according to the present disclosure may also comprise at least one cationic polymer.

As used herein, the term “cationic polymer” is intended to mean any polymer comprising at least one cationic group and/or at least one group which can be ionized to a cationic group.

The cationic polymers which may be used in accordance with the present disclosure may be chosen from all those known in the art to improve the cosmetic properties of hair treated with detergent compositions, for example, those described in European Patent Application No. 0 337 354 and French Patent Application Nos. 2 270 846, 2 383 660, 2 598 611, 2 470 596, and 2 519 863.

In at least one embodiment, the cationic polymers may be chosen from those which comprise units comprising primary, secondary, tertiary, and/or quaternary amine groups which can either be part of the main polymer chain, or can be carried by a side substituent directly connected to the main chain.

The cationic polymers may have a weight-average molecular mass of greater than 10⁵, for instance, greater than 10⁶, or ranging from 10⁶ to 10⁸.

According to one embodiment, the cationic polymers may be chosen from polyamine, polyaminoamide, and quaternary polyammonium polymers, which are known products.

Polyamine, polyamino amide, and quaternary polyammonium polymers that can be used in the composition of the present disclosure are described, for example, in French Patent Nos. 2 505 348 and 2 542 997, for instance:

(1) homopolymers and copolymers derived from acrylic and methacrylic esters and amides and comprising at least one unit chosen from units of the following formulae:

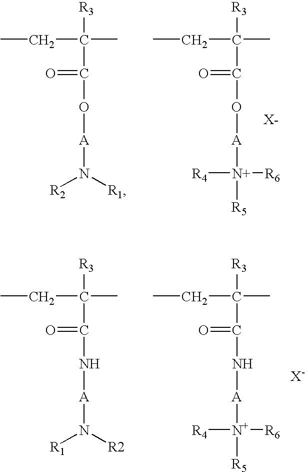

wherein:
R₁ and R₂, which may be identical or different, are chosen from hydrogen and alkyl groups comprising from 1 to 6 carbon atoms, for instance, methyl and ethyl groups;
R₃, which may be identical or different, is chosen from hydrogen and CH₃;
the symbols A, which may be identical or different, are chosen from linear or branched alkyl groups comprising from 1 to 6 carbon atoms, for instance, 2 or 3 carbon atoms, and hydroxyalkyl groups comprising from 1 to 4 carbon atoms;
R₄, R₅, and R₆, which may be identical or different, are chosen from alkyl groups comprising from 1 to 18 carbon atoms and benzyl groups, and in at least one embodiment, alkyl groups comprising from 1 to 6 carbon atoms; and
X is an anion derived from an inorganic or organic acid, such as methosulphate anions and halides, for instance, chloride and bromide.

The copolymers of family (1) may also comprise at least one unit derived from comonomers which may be chosen from the family of acrylamides, methacrylamides, diacetone acrylamides, acrylamides, and methacrylamides substituted on the nitrogen atom with (C₁-C₄) lower alkyl groups, groups derived from acrylic and methacrylic acids and esters thereof, vinyllacatms such as vinylpyrrolidone and vinylcaprolactam, and vinyl esters.

Thus, examples of copolymers of family (1) include, but are not limited to:
copolymers of acrylamide and of dimethylaminoethyl methacrylate quaternized with dimethyl sulphate or with a dimethyl halide,
copolymers of acrylamide and of methacryloyloxyethyltrimethylammonium chloride described, for example, in European Patent Application No. 0 080 976,
copolymers of acrylamide and of methacryloyloxyethyltrimethylammonium methosulphate,
quaternized or nonquaternized vinylpyrrolidone/dialkylaminoalkyl acrylate or methacrylate copolymers, described, for instance, in French Patent Nos. 2 077 143 and 2 393 573,
dimethylaminoethyl methacrylate/vinylcaprolactam/vinylpyrrolidone terpolymers,
vinylpyrrolidone/methacrylamidopropyldimethylamine copolymers, and
quaternized vinylpyrrolidone/dimethylaminopropylmethacrylamide copolymers.

(2) Cellulose ether derivatives comprising quaternary ammonium groups described, for example, in French Patent No. 1 492 597, for instance, the polymers sold under the names “JR” (JR 400, JR 125, JR 30M) and “LR” (LR 400, LR 30M) by the company Union Carbide Corporation. These polymers are also defined in the CTFA dictionary as quaternary ammoniums of hydroxyethylcellulose that have reacted with an epoxide substituted with a trimethylammonium group.

(3) Cationic cellulose derivatives such as cellulose copolymers and cellulose derivatives grafted with a water-soluble monomer of quaternary ammonium, and described, for example, in U.S. Pat. No. 4,131,576, such as hydroxyalkylcelluloses, for instance, hydroxymethyl-, hydroxyethyl-, and hydroxypropylcelluloses grafted, for instance, with a salt chosen from methacryloylethyltrimethylammonium, methacrylamidopropyltrimethylammonium, and dimethyldiallylammonium salts.

Commercial products corresponding to this definition include, for example, the products sold under the name “Celquat® L 200” and “Celquat® H 100” by the company National Starch.

(4) Cationic polysaccharides, such as those described in U.S. Pat. Nos. 3,589,578 and 4,031,307, such as guar gums comprising cationic trialkylammonium groups. Guar gums modified with a salt, for example the chloride, of 2,3-epoxypropyltrimethylammonium may also be used.

Such products are sold, for example, under the trade names JAGUAR® C13 S, JAGUAR® C15, JAGUAR® C17, and JAGUAR® C162 by the company MEYHALL.

(5) Polymers comprising piperazinyl units and divalent alkylene or hydroxyalkylene groups comprising straight or branched chains, optionally interrupted with at least one entity chosen from oxygen, sulphur, nitrogen, aromatic rings, and heterocyclic rings, and also the oxidation and/or quaternization products of these polymers. Such polymers are described, for example, in French Patent Nos. 2 162 025 and 2 280 361.

(6) Water-soluble polyamino amides prepared, for instance, by polycondensation of an acidic compound with a polyamine; these polyamino amides can be crosslinked with at least one entity chosen from epihalohydrins, diepoxides, dianhydrides, unsaturated dianhydrides, bisunsaturated derivatives, bishalohydrins, bisazetidiniums, bishaloacyldiamines, bis-alkyl halides, oligomers resulting from the reaction of a difunctional compound which is reactive with an entity chosen from bishalohydrins, bisazetidiniums, bishaloacyidiamines, bis-alkyl halides, epihalohydrins, diepoxides, and bisunsaturated derivatives; the crosslinking agent being used in an amount ranging from 0.025 to 0.35 mol per amine group of the polyamino amide; these polyamino amides being optionally alkylated or, if they comprise at least one tertiary amine function, they may be quaternized. Such polymers are described, for example, in French Patent Nos. 2 252 840 and 2 368 508.

(7) Polyamino amide derivatives resulting from the condensation of polyalkylene polyamines with polycarboxylic acids, followed by alkylation with difunctional agents, for example, adipic acid/dialkylaminohydroxyalkyldialkylenetriamine polymers in which the alkyl group comprises from 1 to 4 carbon atoms, for instance, methyl, ethyl, and propyl groups, and the alkylene group comprises from 1 to 4 carbon atoms such as ethylene groups. Such polymers are described, for example, in French Patent No. 1 583 363.

In at least one embodiment these derivatives may be chosen from adipic acid/dimethylaminohydroxypropyldiethylenetriamine polymers.

(8) Polymers obtained by reaction of a polyalkylene polyamine comprising two primary amine groups and at least one secondary amine group, with a dicarboxylic acid chosen from diglycolic acid and saturated aliphatic dicarboxylic acids comprising from 3 to 8 carbon atoms. The molar ratio of the polyalkylene polyamine to the dicarboxylic acid may range from 0.8:1 to 1.4:1; the polyamino amide resulting therefrom being reacted with epichlorohydrin in a molar ratio of epichlorohydrin relative to the secondary amine group of the polyamino amide ranging from 0.5:1 and 1.8:1. Such polymers are described, for example, in U.S. Pat. Nos. 3,227,615 and 2,961,347.

(9) Cyclopolymers of alkyldiallylamine and cyclopolymers of dialkyldiallylammonium, such as homopolymers and copolymers comprising, as main constituent of the chain, at least one unit chosen from units of formulas (Va) and (Vb):

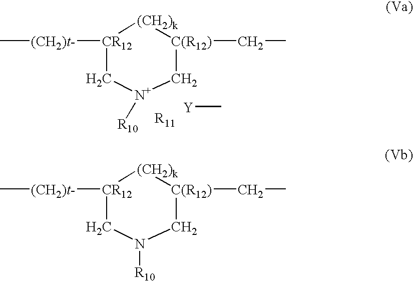

wherein
k and t are equal to 0 or 1, the sum k+t being equal to 1;
R₁₂ is chosen from hydrogen and methyl;
R₁₀ and R₁₁, which may be identical or different, are chosen from alkyl groups comprising from 1 to 6 carbon atoms, hydroxyalkyl groups in which the alkyl group comprises from 1 to 5 carbon atoms, and lower (C₁-C₄)amidoalkyl groups, or alternatively, R₁₀ and R₁₁ may form, together with the nitrogen atom to which they are attached, heterocyclic groups such as piperidinyl and morpholinyl; and
Y⁻ is an anion such as bromide, chloride, acetate, borate, citrate, tartrate, bisulphate, bisulphite, sulphate, and phosphate. These polymers are described, for example, in French Patent No. 2 080 759 and in its Certificate of Addition 2 190 406.

In at least one embodiment, R₁₀ and R₁₁, which may be identical or different, may be chosen from alkyl groups comprising from 1 to 4 carbon atoms.

Further examples of the polymers defined above include, but are not limited to, the dimethyldiallylammonium chloride homopolymer sold under the name “MERQUAT® 100” by the company CALGON (and its homologs of low weight-average molecular mass) and the copolymers of diallyidimethylammonium chloride and of acrylamide sold under the name “MERQUAT® 550”.

(10) Quaternary diammonium polymers comprising at least one repeating unit chosen from those of formula (VI): 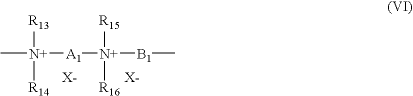
wherein:
R₁₃, R₁₄, R₁₅, and R₁₆, which may be identical or different, are chosen from aliphatic, alicyclic, and arylaliphatic groups comprising from 1 to 20 carbon atoms and lower hydroxyalkyl aliphatic groups, or alternatively, R₁₃, R₁₄, R₁₅, and R₁₆, together or separately, may form, together with the nitrogen atoms to which they are attached, heterocycles optionally comprising a second heteroatom other than nitrogen, or alternatively, R₁₃, R₁₄, R₁₅, and R₁₆ may be chosen from linear or branched C₁-C₆ alkyl groups substituted with at least one group chosen from nitrile groups, ester groups, acyl groups, amide groups, —CO—O—R₁₇-E groups, and —CO—NH—R₁₇-E groups, wherein R₁₇ is an alkylene group and E is a quaternary ammonium group;
A₁ and B₁, which may be identical or different, are chosen from polymethylene groups comprising from 2 to 20 carbon atoms, which may be linear or branched, saturated or unsaturated, and which may comprise, linked or intercalated in the main chain, at least one entity chosen from aromatic rings, oxygen, sulphur, sulphoxide groups, sulphone groups, disulphide groups, amino groups, alkylamino groups, hydroxyl groups, quaternary ammonium groups, ureido groups, amide groups, and ester groups, and
X⁻ is an anion derived from an inorganic or organic acid;
A₁, R₁₃, and R₁₅ may form, together with the two nitrogen atoms to which they are attached, a piperazine ring;
if A₁ is chosen from linear or branched, saturated or unsaturated alkylene or hydroxyalkylene group, B₁ may also be chosen from:
   —(CH₂)ₙ—CO—E′—OC—(CH₂)ₙ—
   wherein E′ is chosen from:

a) glycol residues of formula —O—Z—O—, wherein Z is chosen from linear or branched hydrocarbon-based groups and groups chosen from those of the following formulae:
—(CH₂—CH₂—O)ₓ—CH₂—CH₂—; and
—[CH₂—CH(CH₃)—O]_{y}—CH₂—CH(CH₃)—
wherein x and y, which may be identical or different, are chosen from integers ranging from 1 to 4, representing a defined and unique degree of polymerization, and number ranging from 1 to 4, representing an average degree of polymerization;

b) bis-secondary diamine residues such as piperazine derivatives;

c) bis-primary diamine residues of formula —NH-Y-NH—, wherein Y is chosen from linear or branched hydrocarbon-based groups and divalent groups —CH₂—CH₂—S—S—CH₂—CH₂—; and

d) ureylene groups of formula —NH—CO—NH—.

In at least one embodiment, X⁻ is an anion such as chloride and bromide.

Polymers of this type are described, for example, in French Patent Nos. 2 320 330, 2 270 846, 2 316 271, 2 336 434, and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

Non-limiting examples of such polymers include those comprising at least one repeating unit chosen from those of formula (VII):

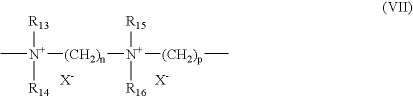

wherein R₁₃, R₁₄, R₁₅, and R₁₆, which may be identical or different, are chosen from alkyl and hydroxyalkyl groups comprising from 1 to 4 carbon atoms, n and p are integers ranging from 2 to 20, and X⁻ is an anion derived from an inorganic or organic acid.

(11) Polyquaternary ammonium polymers comprising at least one unit chosen from those of formula (VIII):

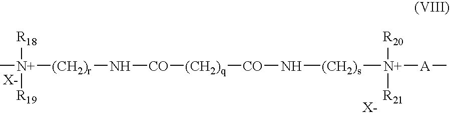

wherein:
R₁₈, R₁₉, R₂₀, and R₂₁, which may be identical or different, are chosen from hydrogen, methyl groups, ethyl groups, propyl groups, β-hydroxyethyl groups, β-hydroxypropyl groups. and —CH₂CH₂(OCH₂CH₂)ₚOH groups, wherein p is chosen from integers ranging from 0 to 6, with the proviso that R₁₈, R₁₉, R₂₀, and R₂₁ are not simultaneously hydrogen,
r and s, which may be identical or different, are integers ranging from 1 to 6,
q is an integer ranging from 1 to 34,
X⁻ is an anion such as a halide, and
A is a radical of a dihalide or —CH₂—CH₂—O—CH₂—CH₂—.

Such compounds are described, for example, in European Patent Application No. 0 122 324.

(12) Quaternary polymers of vinylpyrrolidone and of vinylimidazole.

(13) Crosslinked methacryloyloxy(C₁-C₄)alkyltri(C₁-C₄)alkylammonium salt polymers such as the polymers obtained by homopolymerization of dimethylaminoethyl methacrylate quatemized with methyl chloride, or by copolymerization of acrylamide with dimethylaminoethyl methacrylate quaternized with methyl chloride, the homopolymerization or copolymerization being followed by crosslinking with a compound containing olefinic unsaturation, for example, methylenebisacrylamide.

Other cationic polymers which may be used in the compositions of the present disclosure include cationic proteins and cationic protein hydrolysates, polyalkyleneimines, such as polyethyleneimines, polymers comprising vinylpyridine or vinylpyridinium units, condensates of polyamines and of epichlorohydrin, quaternary polyureylenes, and chitin derivatives.

According to one embodiment, the at least one cationic polymer may be chosen from cellulose ether derivatives comprising quaternary ammonium groups, such as the products sold under the name “JR 400” by the company UNION CARBIDE CORPORATION, cationic cyclopolymers, for example, the homopolymers or copolymers of dimethyldiallylammonium chloride sold under the names MERQUAT® 100, MERQUAT® 550, and MERQUAT® S by the company CALGON, guar gums modified with a 2,3-epoxypropyltrimethylammonium salt, and quaternary polymers of vinylpyrrolidone and of vinylimidazole.

The at least one cationic polymer may be present in the composition in an amount ranging from 0.01 to 10% by weight, for example, from 0.02 to 5% by weight, or from 0.05 to 1% by weight, relative to the total weight of the composition.

The composition according to the present disclosure may also comprise at least one silicone.

The at least one silicone may be soluble or insoluble in the composition. They may be chosen from polyorganosiloxanes that are insoluble in the composition of the present disclosure and may be in a form chosen from oils, waxes, resins, and gums.

The insoluble silicones may be dispersed in the compositions in the form of particles generally having a number-average size ranging from 2 nanometers to 100 micrometers, for example, from 20 nanometers to 20 micrometers (as measured with a particle sizer).

Suitable polyorganosiloxanes are defined, for instance, in the work by Walter NOLL “Chemistry and Technology of Silicones” (1968) Academic Press. These polyorganosiloxanes may be volatile or non-volatile.

When they are volatile, the silicones may be chosen from those having a boiling point ranging from 60° C. to 260° C., for instance:

(i) cyclic silicones comprising from 3 to 7 silicon atoms, for example, 4 or 5, such as the octamethylcyclotetrasiloxane sold under the name “VOLATILE SILICONE 7207” by UNION CARBIDE and “SILBIONE 70045 V 2by RHODIA, the decamethylcyclopentasiloxane sold under the name “VOLATILE SILICONE 7158” by UNION CARBIDE and “SILBIONE 70045 V 5” by RHODIA, and mixtures thereof.

Further examples include, but are not limited to, cyclocopolymers of the dimethylsiloxane/methylalkylsiloxane type, such as the “SILICONE VOLATILE FZ 3109” sold by the company UNION CARBIDE, which has the chemical structure:

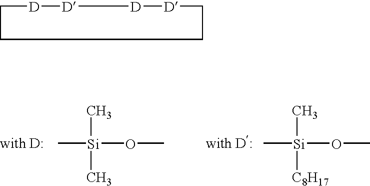

Mixtures of cyclic silicones with silicon-derived organic compounds, such as the mixture of octamethylcyclotetrasiloxane and tetratrimethylsilylpentaerythritol (50/50) and the mixture of octamethylcyclotetrasiloxane and oxy-1,1′-(hexa-2,2,2′,2,3,3′-trimethylsilyloxy)bisneopentane may also be used;

(ii) linear volatile silicones comprising from 2 to 9 silicon atoms and having a viscosity of less than or equal to 5×10⁻⁶ m²/s at 25° C., for example, the decamethyltetrasiloxane sold under the name “SH 200” by the company TORAY SILICONE. Silicones which fall into this class are also described in the article published in Cosmetics and Toiletries, Vol. 91, January 76, p. 27-32—Todd & Byers, “Volatile Silicone Fluids for Cosmetics”.

Non-limiting examples of non-volatile silicones include polyalkylsiloxanes, polyarylsiloxanes, polyalkylarylsiloxanes, silicone gums and resins, polyorganosiloxanes modified with organofunctional groups, polysiloxane(A)-polyoxyalkylene(B) linear block copolymers of (A-B)ₙ type with n>3; grafted silicone polymers, with a non-silicone organic backbone, comprising an organic main chain comprising organic monomers containing no silicone, onto which is grafted, within said chain and also, optionally, on at least one of its ends, at least one polysiloxane macromonomer; grafted silicone polymers, with a polysiloxane backbone grafted with non-silicone organic monomers, comprising a main chain of polysiloxane onto which is grafted, within said chain and also, optionally, on at least one of its ends, at least one organic macromonomer containing no silicone; and mixtures thereof.

Examples of polyalkylsiloxanes include, but are not limited to polydimethylsiloxanes with trimethylsilyl end groups having a viscosity ranging from 5×10^{31 6} to 2.5 m²/s at 25° C., for instance, from 1×10⁻⁵ to 1 m²/s. The viscosity of the silicones is, for example, measured at 25° C. according to ASTM standard 445 Appendix C.

Commercial products corresponding to these polyalkylsiloxanes include, but are not limited to:
the SILBIONE oils of the 47 and 70 047 series or the MIRASIL oils sold by RHONE POULENC, such as, for example, the 70 047 V 500 000 oil;
the oils of the MIRASIL series sold by the company RHONE POULENC;
the oils of the 200 series from the company DOW CORNING, such as DC200 having a viscosity of 60 000 cSt;
the VISCASIL oils from GENERAL ELECTRIC and some oils of the SF series (SF 96, SF 18) from GENERAL ELECTRIC.

Further examples include polydimethylsiloxanes with dimethylsilanol end groups (dimethiconol according to the CTFA name), such as the oils of the 48 series from the company RHONE POULENC.

The products sold under the names “ABIL WAX 9800 and 9801” by the company GOLDSCHMIDT, which are poly(C₁-C₂₀)alkylsiloxanes, also correspond to this class of polyalkylsiloxanes.

The polyalkylarylsiloxanes may also be chosen from linear and/or branched polydimethylmethylphenylsiloxanes and polydimethyldiphenylsiloxanes having a viscosity ranging from 1×10⁻⁵ to 5×10⁻² m²/s at 25° C., for example, the products sold under the following names:
the SILBIONE oils of the 70 641 series from RHONE POULENC;
the oils of RHODORSIL 70 633 and 763 series from RHONE POULENC;
the DOW CORNING 556 COSMETIC GRADE FLUID oil from DOW CORNING;
the silicones of the PK series from BAYER, such as the product PK20;
the silicones of the PN and PH series from BAYER, such as the products PN1000 and PH1000; and
some oils of the SF series from GENERAL ELECTRIC, such as SF 1023, SF 1154, SF 1250, and SF 1265.

The silicone gums which may be used in accordance with the present disclosure may be chosen, for example, from polydiorganosiloxanes having high number-average molecular masses ranging from 200 000 to 1 000 000, used alone or as a mixture in a solvent. This solvent may be chosen from volatile silicones, polydimethylsiloxane (PDMS) oils, polyphenylmethylsiloxane (PPMS) oils, isoparaffins, polyisobutylenes, methylene chloride, pentane, dodecane, tridecanes, and mixtures thereof.

Further examples of suitable silicone gums include, but are not limited to:
polydimethylsiloxane,
polydimethylsiloxane/methylvinylsiloxane gums,
polydimethylsiloxane/diphenylsiloxane,
polydimethylsiloxane/phenylmethylsiloxane, and
polydimethylsiloxane/diphenylsiloxane/methylvinylsiloxane.

Silicone mixtures may also be used, such as:
the mixtures formed from a polydimethylsiloxane hydroxylated at the end of the chain (called dimethiconol according to the nomenclature of the CTFA dictionary) and from a cyclic polydimethylsiloxane (called cyclomethicone according to the nomenclature of the CTFA dictionary), such as the product Q2 1401 sold by the company DOW CORNING;
the mixtures formed from a polydimethylsiloxane gum with a cyclic silicone, such as the product SF 1214 Silicone Fluid from the company GENERAL ELECTRIC; this product is an SF 30 gum corresponding to a dimethicone, having a number-average molecular weight of 500 000 solubilized in the SF 1202 Silicone Fluid oil corresponding to decamethylcyclopentasiloxane; and
the mixtures of two PDMSs having different viscosities, for instance, of a PDMS gum and of a PDMS oil, such as the product SF 1236 from the company GENERAL ELECTRIC. The product SF 1236 is the mixture of an SE 30 gum defined above having a viscosity of 20 m²/s and of an SF 96 oil having a viscosity of 5×10⁻⁶ m²/s. This product may contain 15% of SE 20 gum and 85% of an SF 96 oil.

The organopolysiloxane resins which may be used in accordance with the present disclosure include crosslinked siloxane systems comprising the units: R₂SiO_{2/2}, R₃SiO_{1/2}, RSiO_{3/2}, and SiO_{4/2}, wherein R is chosen from hydrocarbon-based groups comprising from 1 to 16 carbon atoms and phenyl groups.

In at least one embodiment, R may be chosen from C₁-C₄ lower alkyl radicals, such as methyl and phenyl radicals.

Commercial products corresponding to these resins include, but are not limited to, the product sold under the name “DOW CORNING 593” and those sold under the names “SILICONE FLUID SS 4230 and SS 4267” by the company GENERAL ELECTRIC, which are silicones with a dimethyl/trimethylsiloxane structure.

Further non-limiting examples include trimethylsiloxysilicate resins sold, for example, under the names X22-4914, X21-5034, and X21-5037 by the company SHIN-ETSU.

The organomodified silicones which may be used in accordance with the present disclosure include silicones as defined above and comprising, in their structure, at least one organofunctional group attached by means of a hydrocarbon-based group.

The organomodified silicones may include, for example, polyorganosiloxanes comprising:
polyethyleneoxy and/or polypropyleneoxy groups optionally comprising C₆-C₂₄ alkyl groups, such as the products called dimethicone-copolyol sold by the company DOW CORNING under the name DC 1248 and the SILWET® L 722, L 7500, L 77, and L 711 oils from the company UNION CARBIDE and the (C₁₂)alkyl methicone copolyol sold by the company DOW CORNING under the name Q2 5200;
substituted or unsubstituted amino groups, such as the products sold under the name GP 4 Silicone Fluid and GP 7100 by the company GENESEE and the products sold under the names Q2 8220 and DOW CORNING 929 and 939 by the company DOW CORNING. The substituted amino groups may be chosen, for example, from C₁-C₄ aminoalkyl groups;
quaternary ammonium groups, such as the products sold under the names ABILQUAT 3272 and ABILQUAT 3474 by the company GOLDSCHMIDT;
thiol groups, such as the products sold under the names “GP 72 A” and “GP 71” from GENESEE;
alkoxylated groups, such as the product sold under the name “SILICONE COPOLYMER F-755” by SWS SILICONES and ABIL WAX® 2428, 2434 and 2440 by the company GOLDSCHMIDT;
hydroxyl groups, such as the polyorganosiloxanes with a hydroxyalkyl function, described in French Patent Application No. 85 16334;
acyloxyalkyl groups such as the polyorganosiloxanes described in U.S. Pat. No. 4,957,732;
anionic groups of the carboxylic acid type, for example, in the products described in European Patent No. 0 186 507 from the company CHISSO CORPORATION, and of the alkylcarboxylic type such as those present in the product X-22-3701 E from the company SHIN-ETSU; 2-hydroxyalkyl sulphonate; 2-hydroxyalkyl thiosulphate, such as the products sold by the company GOLDSCHMIDT under the names “ABIL® S201” and “ABIL® S255”;
hydroxyacylamino groups, such as the polyorganosiloxanes described in European Patent Application No. 0 342 834, and the product Q2-8413 from the company DOW CORNING.

According to one embodiment, the at least one silicones may be chosen, for example, from polydimethylsiloxanes such as polydimethylsiloxanes with trimethylsilyl end groups, polydimethylsiloxanes with hydroxydimethylsilyl end groups, and aminated silicones.

The at least one silicone may be present in the composition in an amount ranging from 0.05 to 20% by weight, for example, from 0.1 to 10% by weight, or from 0.5 to 5% by weight relative to the total weight of the composition.

The aqueous medium may be chosen from water and mixtures of water and at least one cosmetically acceptable solvent chosen from C₁-C₄ lower alcohols, such as ethanol, isopropanol, tert-butanol, and n-butanol; polyols such as glycerol, propylene glycol, and polyethylene glycols; and mixtures thereof.

The pH of the compositions according to the present disclosure is generally less than 8.5, for example, ranging from 4 to 7.

The composition according to the present disclosure may also comprise at least one conventional additive known in the art, such as anti-hairloss agents, oxidizing agents, ceramides, pseudoceramides, vitamins, provitamins, including panthenol, plant, animal, mineral, and synthetic oils, waxes, sunscreens, colored or non-colored inorganic or organic pigments, dyes, pearlescent agents, opacifiers, sequestering agents, plasticizers, solubilizing agents, acidifying agents, basifying agents, inorganic or organic thickeners, antioxidants, hydroxy acids, fragrances, and preserving agents.

Non-limiting examples of opacifiers include compounds such as hydrostearyl cetyl ether and ethylene glycol distearate.

The composition according to the present disclosure may thus comprise at least one ether having two fatty chains corresponding to formula (I):
R—O—R′ (I)
wherein:
R and R′, which may be identical or different, are chosen from linear or branched alkyl and alkenyl groups comprising from 12 to 30 carbon atoms, for example, from 14 to 24 carbon atoms, R and R′ being chosen in such a way that the compound of formula (I) is solid at a temperature of 30° C. In at least one embodiment, R and R′ are identical and are chosen from alkyl groups such as stearyl.

In another embodiment, the ether having two fatty chains can be distearyl ether, for example, the product sold under the name CUTINA® STE by the company COGNIS.

The at least one ether having two fatty chains may be present in the composition in an amount greater than or equal to 0.5% by weight, for example, from 1 to 5% by weight, or from 1.3 to 2% by weight, relative to the total weight of the composition.

It is to be understood that those skilled in the art will take care to choose the at least one optional additive and the amounts thereof in such a way that they do not harm the properties of the compositions of the present disclosure.

The at least one optional additive may be present in the composition according to the present disclosure in an amount ranging from 0 to 20% by weight relative to the total weight of the composition.

The composition according to the present disclosure may be used, for example, as a composition for the cosmetic treatment and/or care of the hair. In at least one embodiment, it can be used as a shampoo or a composition to be applied before or after a shampoo.

Also disclosed herein is a cosmetic treatment process for the hair which comprises applying to the hair an effective amount of a cosmetic composition as described above.

According to at least one embodiment, such a process comprises applying to the hair an effective amount of the cosmetic composition, and optionally rinsing it out after it has optionally been left on for a period of time.

When the composition according to the present disclosure is applied in the form of a lotion or a cream before or after shampooing, it is optionally left on the hair for a time period ranging from 30 seconds to 5 minutes, and then optionally rinsed out with water.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. In the following examples, all the amounts are indicated as percentage by weight of active material relative to the total weight of the composition, unless otherwise indicated.

### EXAMPLES

### Examples 1 to 3

Three shampoo compositions, in accordance with the present disclosure, were prepared from the ingredients indicated in the table below.

| | Compositions | | |
|---|---|---|---|
| | 1 | 2 | 3 |
| sodium lauryl ether sulphate (2.2 OE) in aqueous | 15.6 | 11.7 | 15.6 |
| solution (TEXAPON N702 from Cognis) | | | |
| cocoylamidopropylbetaine/glyceryl monolaurate in | 2.5 | 1.8 | 2.4 |
| aqueous solution 25/75 (TEGOBETAINE from | | | |
| Goldschmidt) | | | |
| oxyethylene sorbitan monolaurate comprising | 4 | 4 | 4 |
| 4 OE (TWEEN 21 from Uniqema) | | | |
| mixture of 1-(hexadecyloxy)-2-octadecanol/cetyl | 2.5 | 2.5 | 2.5 |
| alcohol 47/53 | | | |
| zinc pyrithione as an aqueous dispersion (ZINC | 1 | 1 | 1 |
| OMADINE PYRITHIONE 48% from Arch Personal | | | |
| Care) | | | |
| polydimethylsiloxane (MW: 250 000) (MIRASIL | 1.5 | 1 | 2.7 |
| DM 500 000 from Rhodia) | | | |
| hexylene glycol (2-methyl-2,4-pentanediol) | 0.8 | 0.5 | |
| sodium chloride | 0.6 | — | — |
| fragrance | 0.5 | 0.5 | 0.5 |
| hydroxyethylcellulose quaternized with | 0.4 | 0.4 | — |
| 2,3-epoxypropyltrimethylammonium chloride | | | |
| (CELQUAT SC 240C from National Starch) | | | |
| coconut acid monoisopropanolamide (EMPILAN | 0.35 | 1.2 | 0.5 |
| CIS from Huntsman) | | | |
| carboxyvinyl polymer synthesized in an ethyl | 0.3 | 0.3 | 0.3 |
| acetate/cyclohexane mixture (CARBOPOL 980 | | | |
| from Noveon) | | | |
| 1,3-dimethylol-5,5-dimethyl hydantoin in an | 0.14 | 0.14 | 0.14 |
| aqueous solution (GLYDANT LTD from Lonza) | | | |
| methyl p-hydroxybenzoate, sodium salt | 0.2 | 0.2 | 0.2 |
| (NIPAGINM SODIUM from Clariant) | | | |
| propylene glycol | 0.1 | 0.1 | 0.1 |
| mixture of methyl, ethyl, propyl, butyl and isobutyl | 0.05 | 0.05 | 0.05 |
| p-hydroxybenzoates and phenoxy-2-ethanol | | | |
| (PHENONIP from Clariant) | | | |
| guar hydroxypropyltrimethylammonium chloride | — | — | 0.05 |
| (JAGUAR C13S from Rhodia) | | | |
| Water qs | 100% | 100% | 100% |

Compositions 1, 2, and 3 in accordance with the present disclosure were found to exhibit an excellent tolerance with respect to the scalp. For example, a very clear decrease in discomforting reactions was observed.

In addition, these compositions exhibited a very good level of anti-dandruff effectiveness, which is at least identical, or even greater than, the conventional compositions which do not contain any oxyethylene sorbitan monolaurate comprising 4 OE.

Finally, these compositions exhibited notable cosmetic properties, such as a very pleasant consistency, and ease in distribution and rinsing out.

After application of these compositions to the hair, and then rinsing, the wet hair was clean and pleasant to the touch. Once dried, the hair was easy to style, and had a very pleasant softness and feel. It was shiny and flexible.

### Examples 4 to 6

Three anti-dandruff shampoo compositions were prepared from the ingredients indicated in the table below. Composition 4 is in accordance with the present disclosure, compositions 5 and 6 are comparative compositions.

| | Compositions | | |
|---|---|---|---|
| | 4 | 5 | 6 |
| sodium lauryl ether sulphate (2.2 OE) in aqueous | 15.4 | 15.4 | 15.4 |
| solution (TEXAPON N702 from Cognis) | | | |
| cocoylamidopropylbetaine/glyceryl monolaurate in | 1.7 | 1.7 | 1.7 |
| aqueous solution 25/75 (TEGOBETAINE from | | | |
| Goldschmidt) | | | |
| oxyethylene sorbitan monolaurate comprising | 6 | — | — |
| 4 OE (TWEEN 21 from Uniqema) | | | |
| oxyethylene sorbitan monopalmitate comprising | — | — | 6 |
| 20 OE (TWEEN 40 from Uniqema) | | | |
| oxyethylene sorbitan monostearate comprising | — | 6 | — |
| 4 OE (TWEEN 61 from Uniqema) | | | |
| ethylene glycol distearate (TEGIN BL 315 from | 2 | 2 | 2 |
| Goldschmidt) | | | |
| sodium N-cocoylamidoethyl, N-ethoxycarboxy- | 0.8 | 0.8 | 0.8 |
| methyl glycinate (MIRANOL C2M conc from | | | |
| Rhodia) | | | |
| polydimethylsiloxane (MIRASIL DM 500 000 from | 1.5 | 1.5 | 1.5 |
| Rhodia) | | | |
| coconut acid monoisopropanolamide (EMPILAN | 0.3 | 0.3 | 0.3 |
| CIS from Huntsman) | | | |
| zinc pyrithione as an aqueous dispersion (ZINC | 1 | 1 | 1 |
| OMADINE PYRITHIONE 48% from Arch Personal | | | |
| Care) | | | |
| sodium benzoate | 0.5 | 0.5 | 0.5 |
| Fragrance | 0.5 | 0.5 | 0.5 |
| mixture of methyl, butyl, ethyl, propyl, isobutyl p- | 0.5 | 0.5 | 0.5 |
| hydroxybenzoates (7/57/22/14) (NIPASTAT from | | | |
| Nipa) | | | |
| hydroxyethylcellulose quaternized with 2,3-epoxy- | 0.4 | 0.4 | 0.4 |
| propyltrimethylammonium chloride (CELQUAT | | | |
| SC 240C from National Starch) | | | |
| powdered salicylic acid | 0.2 | 0.2 | 0.2 |
| carboxyvinyl polymer synthesized in an ethyl | 0.2 | 0.2 | 0.2 |
| acetate/cyclohexane mixture (CARBOPOL 980 | | | |
| from Noveon) | | | |
| methyl p-hydroxybenzoate, sodium salt | 0.03 | 0.03 | 0.03 |
| (NIPAGINM SODIUM from Clariant) | | | |
| Water qs | 100% | 100% | 100% |

Composition 4 according to the present disclosure, as compared with the comparative compositions 5 and 6, exhibited various advantages in terms of use and of viscosity.

First, the oxyethylene sorbitan monostearate comprising 4 OE that was used in comparative composition 5 had to be melted before it was incorporated into the shampoo, which made the preparation of the latter more difficult. Conversely, the oxyethylene sorbitan monolaurate comprising 4 EO that was used in composition 4 in accordance with the present disclosure was liquid at ambient temperature. It was therefore able to be incorporated into the shampoo at ambient temperature, hence a substantial gain in terms of time and energy.

Second, the viscosities of comparative compositions 5 and 6 were not appropriate, as compared with the usual consistency of shampoos. In fact, composition 5 exhibited a flow time at 25° C. of 229 s FC 10 (Ford cup No. 10), which corresponded to a viscosity which was too high (discontinuous flow). Conversely, composition 6 exhibited a flow time at 25° C. of 5 s FC 10, which corresponded to a viscosity which was much too low.

Composition 4 according to the present disclosure had a flow time at 25° C. of 112 s FC 10, which corresponded to a continuous flow and represented a viscosity which is more suitable for a shampoo.

Thus, composition 4 in accordance with the present disclosure exhibited qualities for use that were superior to the comparative compositions: it was neither too thick nor too liquid, thus allowing better distribution of the product on the hair.

What is claimed is:

1. A cosmetic composition comprising, in an aqueous medium, at least one anti-dandruff agent other than 2-pyridone derivatives, and at least one oxyethylenated sorbitan monolaurate comprising from 2 to 25 oxyethylene units.

2. The composition according to claim 1, wherein the at least one anti-dandruff agent is chosen from:
a) pyridinethione salts,
b) trihalocarbamides,
c) triclosan,
d) azole compounds,
e) antifungal polymers,
f) selenium sulphides,
g) sulphur in its various forms, cadmium sulphide, allantoin, coal tar or wood tar and derivatives thereof, salicylic acid, undecylenic acid, fumaric acid, and allylamines.

3. The composition according to claim 2, wherein the pyridinethione salts are chosen from calcium, magnesium, barium, strontium, zinc, cadmium, tin, and zirconium salts.

4. The composition according to claim 2, wherein the azole compounds are chosen from climbazole, ketoconazole, clotrinazole, econazole, isoconazole, and miconazole.

5. The composition according to claim 2, wherein the antifungal polymers are chosen from amphotericin B or nystatin.

6. The composition according to claim 2, wherein the selenium sulphides are chosen from those of formula SₓSe₈₋ₓ, wherein x ranges from 1 to 7,

7. The composition according to claim 2, wherein the at least one anti-dandruff agent is chosen from zinc pyrithione, salicylic acid, selenium disulphide, and mixtures thereof.

8. The composition according to claim 1, wherein the at least one anti-dandruff agent is present in the composition in an amount ranging from 0.001 to 10% by weight relative to the total weight of the composition.

9. The composition according to claim 8, wherein the at least one anti-dandruff agent is present in the composition in an amount ranging from 0.1 to 5% by weight relative to the total weight of the composition.

10. The composition according to claim 9, wherein the at least one anti-dandruff agent is present in the composition in an amount ranging from 0.2 to 2% by weight relative to the total weight of the composition.

11. The composition according to claim 1, wherein the at least one oxyethylenated sorbitan monolaurate is chosen from those comprising from 2 to 10 oxyethylene units.

12. The composition according to claim 11, wherein the at least one oxyethylene sorbitan monolaurate comprises 4 OE.

13. The composition according to claim 1, wherein the at least one oxyethylenated sorbitan monolaurate comprising from 2 to 25 oxyethylene units is present in the composition in an amount of at least 0.5% by weight relative to the total weight of the composition.

14. The composition according to claim 13, wherein the at least one oxyethylenated sorbitan monolaurate comprising from 2 to 25 oxyethylene units is present in the composition in an amount ranging from 0.5 to 10% by weight relative to the total weight of the composition.

15. The composition according to claim 14, wherein the at least one oxyethylenated sorbitan monolaurate comprising from 2 to 25 oxyethylene units is present in the composition in an amount ranging from 2 to 9% by weight relative to the total weight of the composition.

16. The composition according to claim 1, further comprising at least one ether having two fatty chains, chosen from ethers having two fatty chains which are solid at a temperature of 30° C., corresponding to formula (I):
R—O—R′ (I)
wherein:
R and R′, which may be identical or different, are chosen from linear or branched alkyl and alkenyl groups comprising from 12 to 30 carbon atoms, R and R′ being chosen such that the compound of formula (I) is solid at a temperature of 30° C.

17. The composition according to claim 16, wherein the at least one ether having two fatty chains is distearyl ether.

18. The composition according to claim 16, wherein the at least one ether having two fatty chains is present in the composition in an amount greater than or equal to 0.5% by weight relative to the total weight of the composition.

19. The composition according to claim 18, wherein the at least one ether having two fatty chains is present in the composition in an amount ranging from 1 to 5% by weight relative to the total weight of the composition.

20. The composition according to claim 19, wherein the at least one ether having two fatty chains is present in the composition in an amount ranging from 1.3 to 2% by weight relative to the total weight of the composition.

21. The composition according to claim 1, further comprising at least one fatty alcohol comprising at least 12 carbon atoms.

22. The composition according to claim 21, wherein the at least one fatty alcohol comprises from 12 to 30 carbon atoms.

23. The composition according to claim 21, wherein the at least one fatty alcohol is chosen from cetyl alcohol, stearyl alcohol, arachidyl alcohol, behenyl alcohol, lignoceryl alcohol, ceryl alcohol, montanyl alcohol, and mixtures thereof.

24. The composition according to claim 21, wherein the at least one fatty alcohol is present in the composition in an amount greater than or equal to 0.5% by weight relative to the total weight of the composition.

25. The composition according to claim 24, wherein the at least one fatty alcohol is present in the composition in an amount ranging from 0.5 to 10% by weight relative to the total weight of the composition.

26. The composition according to claim 25, wherein the at least one fatty alcohol is present in the composition in an amount ranging from 1 to 5% by weight relative to the total weight of the composition.

27. The composition according to claim 1, further comprising at least one surfactant chosen from anionic surfactants, cationic surfactants, amphoteric or zwitterionic surfactants, and nonionic surfactants different from the oxyethylenated sorbitan monolaurate comprising from 2 to 25 oxyethylene units.

28. The composition according to claim 27, wherein the composition comprises at least one anionic surfactant and at least one amphoteric or zwitterionic surfactant.

29. The composition according to claim 27, wherein the at least one anionic surfactant is chosen from alkyl sulphates, alkyl ether sulphates, and mixtures thereof.

30. The composition according to claim 27 wherein the at least one anionic surfactant is present in the composition in an amount ranging from 0.1 to 50% by weight relative to the total weight of the composition.

31. The composition according to claim 30, wherein the at least one anionic surfactant is present in the composition in an amount ranging from 4 to 20% by weight relative to the total weight of the composition.

32. The composition according to claim 27, wherein the at least one amphoteric or zwitterionic surfactant is chosen from (C₈₋₂₀ alkyl)betaines, (C₈₋₂₀ alkyl)amino(C₆₋₈ alkyl)betaines, and mixtures thereof.

33. The composition according to claim 1, further comprising at least one cationic polymer.

34. The composition according to claim 1, further comprising at least one silicone.

35. The composition according to claim 1, wherein the aqueous medium is chosen from water and mixtures of water and at least one cosmetically acceptable solvent.

36. The composition according to claim 35, wherein the at least one solvent is chosen from C₁-C₄ lower alcohols and polyols.

37. The composition according to claim 1, further comprising at least one additive chosen from anti-hairloss agents, oxidizing agents, ceramides, pseudo-ceramides, vitamins, provitamins, plant, animal, mineral, and synthetic oils, waxes, sunscreens, colored or non-colored inorganic or organic pigments, dyes, pearlescent agents, opacifiers, sequestering agents, plasticizers, solubilizing agents, acidifying agents, basifying agents, inorganic or organic thickeners, antioxidants, hydroxy acids, fragrances, and preserving agents.

38. A process for the cosmetic treatment and/or care of hair, said process comprising applying to the hair an effective amount of a cosmetic composition comprising, in an aqueous medium, at least one anti-dandruff agent other than 2-pyridone derivatives, and at least one oxyethylenated sorbitan monolaurate comprising from 2 to 25 oxyethylene units.

39. The process according to claim 38, wherein the cosmetic composition is chosen from shampoos and compositions to be applied before or after a shampoo.
